(19) United States
(12) Patent Application Publication
(10) Pub. No.: US 2025/0128069 A1
(43) Pub. Date: Apr. 24, 2025

(54) ENHANCED SYSTEMS AND METHODS FOR DATA CORRECTION IN SENSED NEUROMODULATION APPLICATIONS

(71) Applicant: Boston Scientific Neuromodulation Corporation, Valencia, CA (US)

(72) Inventors: Andrew James Haddock, Los Angeles, CA (US); G. Karl Steinke, Valencia, CA (US); Raul Enrique Serrano Carmona, Los Angeles, CA (US)

(21) Appl. No.: 18/905,879

(22) Filed: Oct. 3, 2024

Related U.S. Application Data
(60) Provisional application No. 63/544,977, filed on Oct. 20, 2023.

(51) Int. Cl. A61N 1/36; A61N 1/372

(52) U.S. Cl. CPC A61N 1/36139; A61N 1/37223; A61N 1/37235

(57) ABSTRACT

This document discusses a computer-implemented method of operating a neurostimulation device to deliver electrical neurostimulation when connected to an implantable stimulation lead. The method includes delivering neurostimulation to a subject using a neurostimulation device of the neurostimulation system when the neurostimulation device is connected to an implantable stimulation lead; recording electrical signals sensed using the implantable stimulation lead; detecting and correcting errors in a recorded electrical signal to produce a corrected recorded electrical signal; and adjusting a device-based neurostimulation therapy provided to the subject using the corrected recorded electrical signal.

### CLAIM OF PRIORITY

This application claims the benefit of U.S. Provisional Application No. 63/544,977, filed on Oct. 20, 2023, which is hereby incorporated by reference in its entirety.

### TECHNICAL FIELD

This document relates generally to medical devices and more particularly to a system for neurostimulation.

### BACKGROUND

Neurostimulation, also referred to as neuromodulation, has been proposed as a therapy for a number of conditions. Examples of neurostimulation include Spinal Cord Stimulation (SCS), Deep Brain Stimulation (DBS), Peripheral Nerve Stimulation (PNS), and Functional Electrical Stimulation (FES). Implantable neurostimulation systems have been applied to deliver such a therapy. An implantable neurostimulation system may include an implantable neurostimulator, also referred to as an implantable pulse generator (IPG), and one or more implantable leads each including one or more electrodes. The implantable neurostimulator delivers neurostimulation energy through one or more electrodes placed on or near a target site in the nervous system. An external programming device can be used to program the implantable neurostimulator with stimulation parameters controlling the delivery of the neurostimulation energy.

In one example, the neurostimulation energy is delivered in the form of electrical neurostimulation pulses. The delivery is controlled using stimulation parameters that specify spatial (where to stimulate), temporal (when to stimulate), and informational (patterns of pulses directing the nervous system to respond as desired) aspects of a pattern of neurostimulation pulses. Neurostimulation systems may offer many programmable options for the parameters of the neurostimulation to customize the neurostimulation therapy for a specific patient. For some types of neurostimulation (e.g., DBS) the efficacy of the neurostimulation for the patient may depend on the neurostimulation system performing adaptive or decision-making algorithms to optimize the neurostimulation therapy. The decision-making algorithms performed by the neurostimulation system may rely on accurate data to output a meaningful and correct therapy decision. The decision-making performance of the neurostimulation system can be compounded by system-level problems that may affect data fidelity. Basing decisions regarding neurostimulation therapy using signals that include missing or incorrect data can lead to patient dissatisfaction with the neurostimulation.

### SUMMARY

Electrical neurostimulation energy can be delivered in the form of electrical neurostimulation pulses to treat a neurological condition of the patient. The pulses can be delivered using an implantable stimulation lead. The lead can have multiple electrodes and may be configurable into many electrode configurations. Neurostimulation can be provided to the patient and electrical signals evoked by the neurostimulation can be sensed, recorded, and analyzed to customize the neurostimulation to a particular patient. However, the recording can include data errors such as incorrect data or missing data.

Example 1 includes subject matter (such as a method of operating a neurostimulation system) comprising delivering neurostimulation to a subject using a neurostimulation device of the neurostimulation system when the neurostimulation device is connected to an implantable stimulation lead, recording electrical signals sensed using the implantable stimulation lead, detecting and correcting errors in a recorded electrical signal to produce a corrected recorded electrical signal, and adjusting a device-based neurostimulation therapy provided to the subject using the corrected recorded electrical signal.

In Example 2, the subject matter of Example 1 optionally includes at least one of the neurostimulation device and a programmer of the neurostimulation device detecting incorrect data and removing the incorrect data from the recorded electrical signal.

In Example 3, the subject matter of Example 2 optionally includes the at least one of the neurostimulation device and the programmer applying a signal smoothing algorithm to the recorded electrical signal.

In Example 4, the subject matter of one or any combination of Examples 1-3 optionally includes at least one of the neurostimulation device and a programmer of the neurostimulation device detecting missing data in the recorded electrical signal and producing the missing data using a data interpolation algorithm.

In Example 5, the subject matter of Example 4 optionally includes the at least one of the neurostimulation device and the programmer producing the missing data using one or more of an expectation-maximization (EM) algorithm, a maximum-likelihood estimation (MLE) algorithm, a Kalman smoothing algorithm, or low pass filtering of the recorded electrical signal.

In Example 6, the subject matter of one or any combination of Examples 1-5 optionally includes filtering the corrected recorded electrical signal to produce a filtered corrected recorded electrical signal, and detecting and correcting a signal artifact in the filtered corrected recorded electrical signal introduced by the filtering.

In Example 7, the subject matter of one or any combination of Examples 1-6 optionally includes extracting one or more features from the corrected recorded electrical signal, detecting an error in the one or more extracted features of the corrected recorded electrical signal, and removing the one or more extracted features having the detected error or correcting the error in the one or more extracted features.

In Example 8, the subject matter of one or any combination of Examples 1-7 optionally includes extracting one or more features from the corrected recorded electrical signal, and identifying an evoked response signal of interest from among the recorded electrical signals using the one or more extracted features of the corrected recorded electrical signal.

Example 9 includes subject matter (such as a programming device for a neurostimulation device that provides electrical neurostimulation) or can optionally be combined with one or any combination of Examples 1-8 to include such subject matter, comprising a communication circuit and signal processing circuitry. The communication circuit is configured to receive a recorded electrical signal from the neurostimulation device and send the at least one therapy parameter to the neurostimulation device. The signal processing circuitry is configured to detect and correct errors in the recorded electrical signal to produce a corrected recorded electrical signal, and determine a value of the at least one therapy parameter using the corrected recorded electrical signal.

In Example 10, the subject matter of Example 9 optionally includes signal processing circuitry configured to detect incorrect data in the recorded electrical signal and remove the incorrect data from the recorded electrical signal to produce the corrected recorded electrical signal.

In Example 11, the subject matter of Example 10 optionally includes signal processing circuitry configured to perform a signal smoothing algorithm on the recorded electrical signal to produce the corrected recorded electrical signal.

In Example 12, the subject matter of one or any combination of Examples 9-11 optionally includes signal processing circuitry configured to detect missing data in the recorded electrical signal and perform a data interpolation algorithm to include the missing data in the corrected recorded electrical signal.

In Example 13, the subject matter of Example 12 optionally includes signal processing circuitry configured to perform at least one of an expectation-maximization (EM) algorithm, a maximum-likelihood estimation (MLE) algorithm, a Kalman smoothing algorithm, or low pass filtering of the recorded electrical signal.

In Example 14, the subject matter of one or any combination of Examples 9-13 optionally includes signal processing circuitry configured to filter the corrected recorded electrical signal to produce a filtered corrected recorded electrical signal and detect and correct a signal artifact in the filtered corrected recorded electrical signal introduced by the filtering.

In Example 15, the subject matter of one or any combination of Examples 9-14 optionally includes signal processing circuitry configured to extract one or more features from the corrected recorded electrical signal, detect an error in the one or more extracted features of the corrected recorded electrical signal, and remove the one or more extracted features having the detected error or correct the error in the one or more extracted features.

In Example 16, the subject matter of one or any combination of Examples 9-15 optionally includes signal processing circuitry configured to extract one or more features from the corrected recorded electrical signal, and identify an evoked response signal of interest from among the recorded electrical signals using the one or more extracted features of the corrected recorded electrical signal.

Example 17 includes subject matter (such as a neurostimulation device) or can optionally be combined with one or any combination of Examples 1-16 to include such subject matter, comprising a stimulation circuit configured to deliver electrical neurostimulation to a subject when coupled to an implantable stimulation lead, a sensing circuit configured to sense electrical signals when coupled to the stimulation lead, and a control circuit operatively coupled to the stimulation circuit and the sensing circuit. The control circuit is configured to initiate delivery of neurostimulation to the subject and record sensed electrical signals resulting from the neurostimulation. The device also includes signal processing circuitry configured to detect and correct errors in a recorded electrical signal to produce a corrected recorded electrical signal. The control circuit is further configured to adjust a device-based neurostimulation therapy provided to the subject using the corrected recorded electrical signal.

In Example 18, the subject matter of Example 17 optionally includes signal processing circuitry configured to detect incorrect data in the recorded electrical signal and remove the incorrect data from the recorded electrical signal to produce the corrected recorded electrical signal.

In Example 19, the subject matter of one or both of Examples 17 and 18 optionally includes signal processing circuitry configured to detect missing data in the recorded electrical signal and perform a data interpolation algorithm to include the missing data in the corrected recorded electrical signal.

In Example 20, the subject matter of one or any combination of Examples 17-19 optionally includes signal processing circuitry configured to extract one or more features from the corrected recorded electrical signal, and identify an evoked response signal of interest from among the recorded electrical signals using the one or more extracted features of the corrected recorded electrical signal.

These non-limiting examples can be combined in any permutation or combination. This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is an illustration of portions of an example of a neurostimulation system.

FIG. 2 is an illustration of portions of another example of a neurostimulation system.

FIG. 3 is an illustration of an example of an implantable pulse generator (IPG) and an implantable lead system.

FIG. 4 is an illustration of another example of an IPG and an implantable lead system.

FIG. 5 is a schematic side view of an example of an electrical stimulation lead.

FIGS. 6A-6H are illustrations of an example of electrodes of a stimulation lead.

FIG. 7 is a block diagram of portions of an example of a neurostimulation system.

FIG. 8 is a block diagram of portions of another example of a neurostimulation system.

FIG. 9 is a flow diagram of an example of a method to operate a neurostimulation system to deliver electrical neurostimulation.

FIG. 10 is an illustration of an example of an electrical signal recorded by the neurostimulation system.

FIG. 11 shows an example of a transfer function of an analog-to-digital converter (ADC).

FIG. 12 is a flow diagram of another example of a method to operate a neurostimulation system to deliver electrical neurostimulation.

FIGS. 13 and 14 illustrate examples of a hotspot view that can be produced by a neurostimulation system.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural, logical and electrical changes may be made without departing from the spirit and scope of the present invention. References to “an”, “one”, or “various” embodiments in this disclosure are not necessarily to the same embodiment, and such references contemplate more than one embodiment. The following detailed description provides examples, and the scope of the present invention is defined by the appended claims and their legal equivalents.

This document discusses devices, systems and methods for programming and delivering electrical neurostimulation to a patient or subject. Advancements in neuroscience and neurostimulation research have led to a demand for delivering complex patterns of neurostimulation energy for various types of therapies. The present system may be implemented using a combination of hardware and software designed to apply any neurostimulation (neuromodulation) therapy, including but not being limited to DBS, SCS, PNS, FES, Occipital Nerve Stimulation (ONS), Sacral Nerve Stimulation (SNS), and Vagus Nerve Stimulation (VNS) therapies.

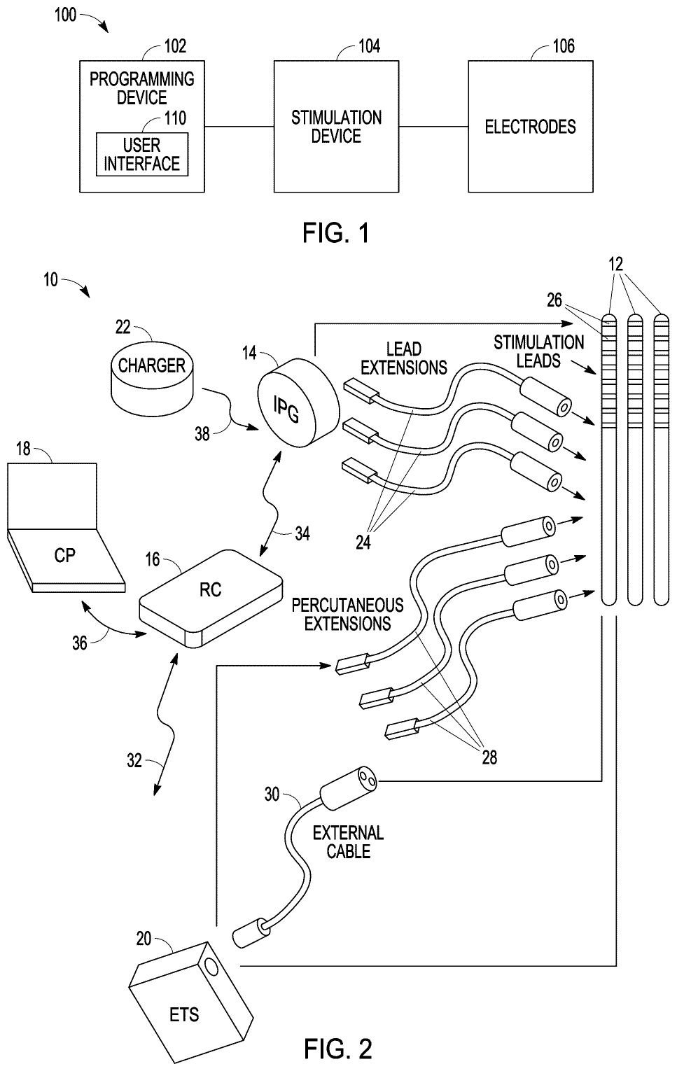

FIG. 1 illustrates an example of portions of a neurostimulation system 100. System 100 includes electrodes 106, a stimulation device 104, and a programming device 102. Electrodes 106 are configured to be placed on or near one or more neural targets in a patient. Stimulation device 104 is configured to be electrically connected to electrodes 106 and deliver neurostimulation energy, such as in the form of electrical pulses, to the one or more neural targets though electrodes 106. The delivery of the neurostimulation is controlled by using multiple stimulation parameters, such as stimulation parameters specifying a pattern of the electrical pulses and a selection of electrodes through which each of the electrical pulses is delivered. In various embodiments, at least some of the stimulation parameters are programmable by a user, such as a physician or other caregiver who treats the patient using system 100. Programming device 102 provides the user with accessibility to the user-programmable parameters. In various embodiments, programming device 102 is configured to be communicatively coupled to stimulation device 104 via a wired or wireless link.

In this document, a “user” includes a physician or other clinician or caregiver who treats the patient using system 100; a “patient” includes a person who receives or is intended to receive neurostimulation delivered using system 100. In various embodiments, the patient can be allowed to adjust their treatment using system 100 to certain extent, such as by adjusting certain therapy parameters and entering feedback and clinical effect information.

In various embodiments, programming device 102 can include a user interface 110 that allows the user to control the operation of system 100 and monitor the performance of system 100 as well as conditions of the patient including responses to the delivery of the neurostimulation. The user can control the operation of system 100 by setting and/or adjusting values of the user-programmable parameters.

In various embodiments, user interface 110 can include a graphical user interface (GUI) that allows the user to set and/or adjust the values of the user-programmable parameters by creating and/or editing graphical representations of various stimulation waveforms. Such waveforms may include, for example, a waveform representing a pattern of neurostimulation pulses to be delivered to the patient as well as individual waveforms that are used as building blocks of the pattern of neurostimulation pulses, such as the waveform of each pulse in the pattern of neurostimulation pulses. The GUI may also allow the user to set and/or adjust stimulation fields each defined by a set of electrodes through which one or more neurostimulation pulses represented by a waveform are delivered to the patient. The stimulation fields may each be further defined by the distribution of the current of each neurostimulation pulse in the waveform. In various embodiments, neurostimulation pulses for a stimulation period (such as the duration of a therapy session) may be delivered to multiple stimulation fields.

In various embodiments, system 100 can be configured for neurostimulation applications. User interface 110 can be configured to allow the user to control the operation of system 100 for neurostimulation. For example, system 100 as well as user interface 110 can be configured for DBS applications. The DBS configurations include various features that may simplify the task of the user in programming the stimulation device 104 for delivering DBS to the patient, such as the features discussed in this document.

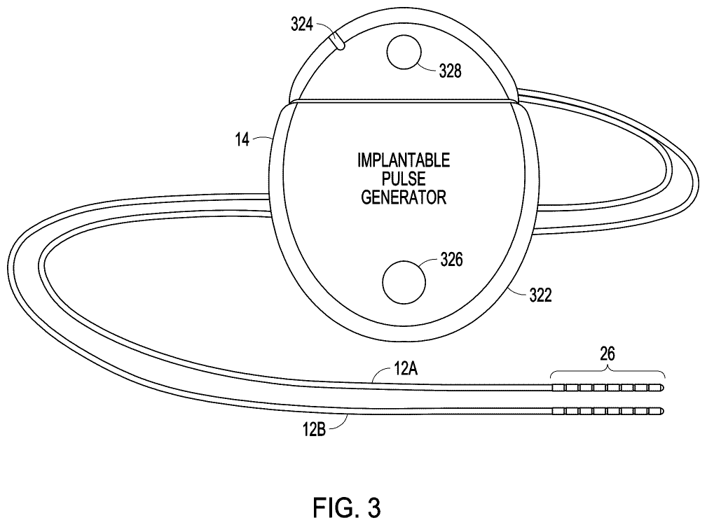

FIG. 2 is an illustration of portions of another example of a neurostimulation system 10 that includes one or more stimulation leads 12 and an implantable pulse generator (IPG) 14. The system 10 can also include one or more of an external remote control (RC) 16, a clinician's programmer (CP) 18, an external trial stimulator (ETS) 20, or an external charger 22. The IPG 14 can optionally be physically connected via one or more lead extensions 24, to the stimulation lead(s) 12. Each lead carries multiple electrodes 26 arranged in an array. The IPG 14 includes pulse generation circuitry that delivers electrical stimulation energy in the form of, for example, a pulsed electrical waveform (i.e., a temporal series of electrical pulses) to the electrode array 26 in accordance with a set of stimulation parameters. The IPG 14 can be implanted into a patient's body, for example, below the patient's clavicle area or within the patient's buttocks or abdominal cavity. The implantable pulse generator can have multiple stimulation channels (e.g., 8 or 16) which may be independently programmable to control the magnitude of the current stimulus from each channel. The IPG 14 can have one, two, three, four, or more connector ports, for receiving the terminals of the leads 12.

The ETS 20 may also be physically connected, optionally via the percutaneous lead extensions 28 and external cable 30, to the stimulation leads 12. The ETS 20, which may have similar pulse generation circuitry as the IPG 14, can also deliver electrical stimulation energy in the form of, for example, a pulsed electrical waveform to the electrode array 26 in accordance with a set of stimulation parameters. One difference between the ETS 20 and the IPG 14 is that the ETS 20 is often a non-implantable device that is used on a trial basis after the neurostimulation leads 12 have been implanted and prior to implantation of the IPG 14, to test the responsiveness of the stimulation that is to be provided. Any functions described herein with respect to the IPG 14 can likewise be performed with respect to the ETS 20.

The RC 16 may be used to telemetrically communicate with or control the IPG 14 or ETS 20 via a wireless communications link 32. Once the IPG 14 and neurostimulation leads 12 are implanted, the RC 16 may be used to telemetrically communicate with or control the IPG 14 via communications link 34. The communication or control allows the IPG 14 to be turned on or off and to be programmed with different stimulation parameter sets. The IPG 14 may also be operated to modify the programmed stimulation parameters to actively control the characteristics of the electrical stimulation energy output by the IPG 14. The CP 18 allows a user, such as a clinician, the ability to program stimulation parameters for the IPG 14 and ETS 20 in the operating room and in follow-up sessions. The CP 18 may perform this function by indirectly communicating with the IPG 14 or ETS 20, through the RC 16, via a wireless communications link 36. Alternatively, the CP 18 may directly communicate with the IPG 14 or ETS 20 via a wireless communications link (not shown). The stimulation parameters provided by the CP 18 are also used to program the RC 16, so that the stimulation parameters can be subsequently modified by operation of the RC 16 in a stand-alone mode (i.e., without the assistance of the CP 18).

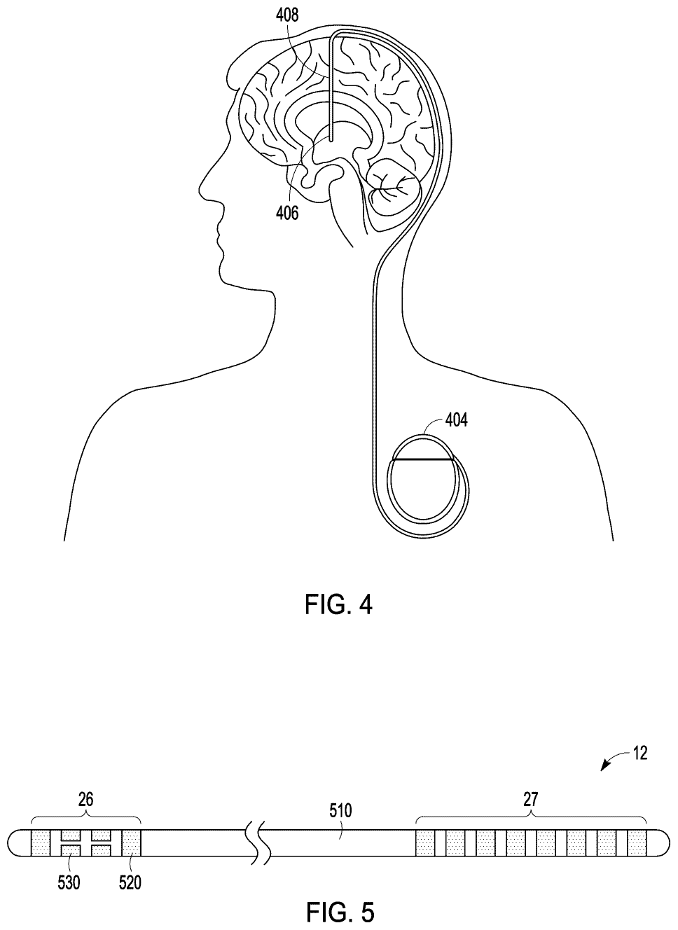

FIG. 3 is an illustration of an example of an IPG 14 (e.g., IPG 14 in FIG. 2) and an implantable lead system that includes stimulation leads (e.g., stimulation leads 12 in FIG. 2). The IPG 14 can be used as stimulation device 104 in FIG. 1. As illustrated in FIG. 3, IPG 14 that can be coupled to implantable leads 12A and 12B at a proximal end of each lead. The distal end of each lead includes electrical contacts or electrodes 26 for contacting a tissue site targeted for electrical neurostimulation. As illustrated in FIG. 3, leads 12A and 12B each include 8 electrodes 26 at the distal end. The number and arrangement of leads 12A and 12B and electrodes 26 as shown in FIGS. 2 and 3 are only examples, and other numbers and arrangements are possible. In various examples, the lead electrodes 26 are ring electrodes. In various examples the lead electrodes 26 include one or more segmented electrodes.

The IPG 14 can include a hermetically sealed IPG case 322 to house the electronic circuitry of IPG 14. IPG 14 can include an electrode 326 formed on IPG case 322. IPG 14 can include an IPG header 324 for coupling the proximal ends of leads 12A and 12B. IPG header 324 may optionally also include an electrode 328. One or both of electrodes 326 and 328 may be used as a reference electrode.

The implantable leads and electrodes may be configured by shape and size to provide electrical neurostimulation energy to a neuronal target included in the subject's brain. Neurostimulation energy can be delivered in a monopolar (also referred to as unipolar) mode using electrode 326 or electrode 328 and one or more electrodes selected from electrodes 26. Neurostimulation energy can be delivered in a bipolar mode using a pair of electrodes of the same lead (lead 12A or lead 12B). Neurostimulation energy can be delivered in an extended bipolar mode using one or more electrodes of a lead (e.g., one or more electrodes of lead 12A) and one or more electrodes of a different lead (e.g., one or more electrodes of lead 12B).

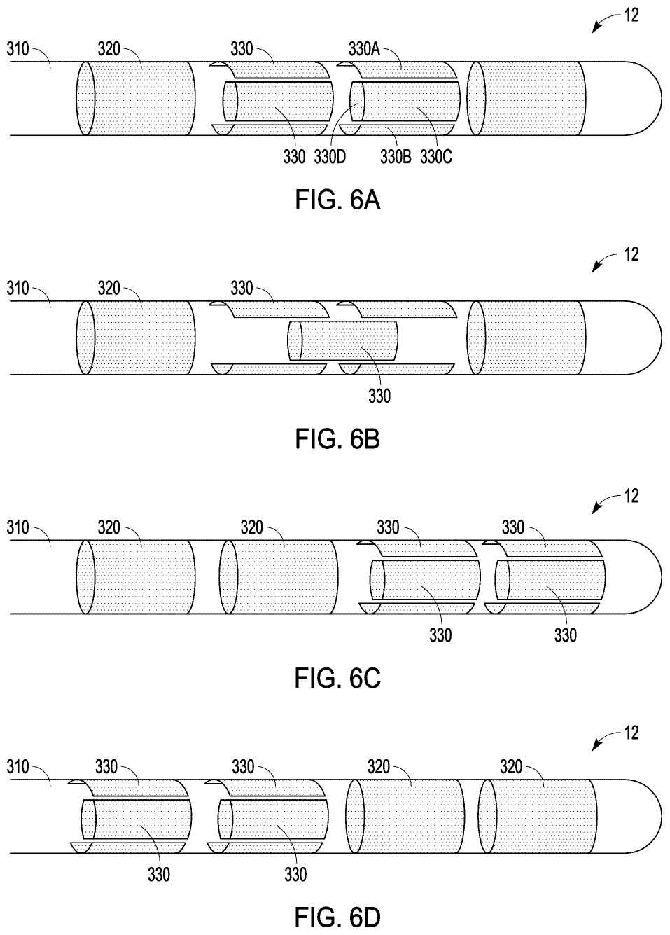

FIG. 4 illustrates another example of an IPG 404 and an implantable lead system 408 arranged to provide neurostimulation to a patient. An example of IPG 404 includes IPG 14 of FIGS. 2 and 3. An example of lead system 408 includes one or more of leads 12A and 12B in FIG. 3. The distal end 406 of the lead includes multiple electrodes (e.g., electrodes 26 in FIG. 3). In the illustrated embodiment, implantable lead system 408 is arranged to provide Deep Brain Stimulation (DBS) to a patient, with the stimulation target being neuronal tissue in a subdivision of the thalamus of the patient's brain. Other examples of DBS targets include neuronal tissue of the globus pallidus (GPi), the subthalamic nucleus (STN), the pedunculopontine nucleus (PPN), substantia nigra pars reticulate (SNr), cortex, globus pallidus externus (GPe), medial forebrain bundle (MFB), periaquaductal gray (PAG), periventricular gray (PVG), habenula, subgenual cingulate, ventral intermediate nucleus (VIM), anterior nucleus (AN), other nuclei of the thalamus, zona incerta, ventral capsule, ventral striatum, nucleus accumbens, and any white matter tracts connecting these and other structures.

After implantation, a clinician will program the neurostimulation device 400 using a CP 18, remote control, or other programming device. The programmed neurostimulation device 400 can be used to treat a neurological condition of the patient, such as Parkinson's Disease, Tremor, Epilepsia, Alzheimer's Disease, other Dementias, Stroke, Multiple Sclerosis, Amyotrophic Lateral Sclerosis (ALS), Autism, brain injury, brain tumor, migraine or other pain or headache condition, and any neurological syndromes that are congenic, degenerative, or acquired.

Returning to FIG. 3, the electronic circuitry of IPG 14 can include a stimulation control circuit that controls delivery of the neurostimulation energy. The stimulation control circuit can include a microprocessor, a digital signal processor, application specific integrated circuit (ASIC), or other type of processor, interpreting or executing instructions included in software or firmware. The neurostimulation energy can be delivered according to specified (e.g., programmed) modulation parameters. Examples of setting modulation parameters can include, among other things, selecting the electrodes or electrode combinations used in the stimulation, configuring an electrode or electrodes as the anode or the cathode for the stimulation, specifying the percentage of the neurostimulation provided by an electrode or electrode combination, and specifying stimulation pulse parameters. Examples of pulse parameters include, among other things, the amplitude of a pulse (specified in current or voltage), pulse duration (e.g., in microseconds), pulse rate (e.g., in pulses per second), and parameters associated with a pulse train or pattern such as burst rate (e.g., an “on” modulation time followed by an “off” modulation time), amplitudes of pulses in the pulse train, polarity of the pulses, etc.

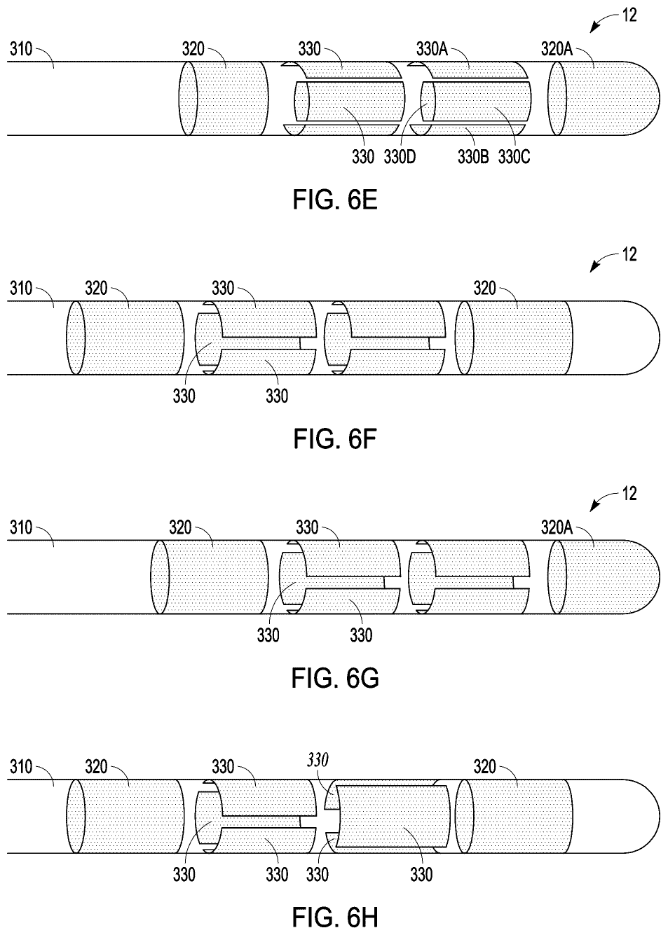

FIG. 5 is a schematic side view of an embodiment of an electrical stimulation lead. FIG. 5 illustrates a stimulation lead 12 with electrodes 26 disposed at least partially about a circumference of the lead 12 along a distal end portion of the lead and terminals 27 disposed along a proximal end portion of the lead. The lead 12 can be implanted near or within the desired portion of the body to be stimulated (e.g., the brain, spinal cord, or other body organs or tissues). In one example of operation for deep brain stimulation, access to the desired position in the brain can be accomplished by drilling a hole in the patient's skull or cranium with a cranial drill (commonly referred to as a burr), and coagulating and incising the dura mater, or brain covering. The lead 12 can be inserted into the cranium and brain tissue with the assistance of a stylet (not shown). The lead 12 can be guided to the target location within the brain using, for example, a stereotactic frame and a microdrive motor system. In some embodiments, the microdrive motor system can be fully or partially automatic. The microdrive motor system may be configured to perform one or more the following actions (alone or in combination): insert the lead 12, advance the lead 12, retract the lead 12, or rotate the lead 12.

In some embodiments, measurement devices coupled to the muscles or other tissues stimulated by the target neurons, or a unit responsive to the patient or clinician, can be coupled to the implantable pulse generator or microdrive motor system. The measurement device, user, or clinician can indicate a response by the target muscles or other tissues to the stimulation or recording electrode(s) to further identify the target neurons and facilitate positioning of the stimulation electrode(s). For example, if the target neurons are directed to a muscle experiencing tremors, a measurement device can be used to observe the muscle and indicate changes in, for example, tremor frequency or amplitude in response to stimulation of neurons. Alternatively, the patient or clinician can observe the muscle and provide feedback.

The lead 12 for deep brain stimulation can include stimulation electrodes, recording electrodes, or both. In at least some embodiments, the lead 12 is rotatable so that the stimulation electrodes can be aligned with the target neurons after the neurons have been located using the recording electrodes. Stimulation electrodes may be disposed on the circumference of the lead 12 to stimulate the target neurons. Stimulation electrodes may be ring-shaped so that current projects from each electrode equally in every direction from the position of the electrode along a length of the lead 12. In the embodiment of FIG. 5, two of the electrodes 520 are ring electrodes 520. Ring electrodes typically do not enable stimulus current to be directed from only a limited angular range around of the lead. Segmented electrodes 530, however, can be used to direct stimulus current to a selected angular range around the lead. When segmented electrodes 530 are used in conjunction with an IPG 14 that delivers constant current stimulus, current steering can be achieved to more precisely deliver the stimulus to a position around an axis of the lead (e.g., radial positioning around the axis of the lead). To achieve current steering, segmented electrodes can be utilized in addition to, or as an alternative to, ring electrodes.

The lead 12 includes a lead body 510, terminals 27, and one or more ring electrodes 520 and one or more sets of segmented electrodes 530 (or any other combination of electrodes). The lead body 510 can be formed of a biocompatible, non-conducting material such as, for example, a polymeric material. Suitable polymeric materials include, but are not limited to, silicone, polyurethane, polyurea, polyurethaneurea, polyethylene, or the like. Once implanted in the body, the lead 12 may be in contact with body tissue for extended periods of time. In at least some embodiments, the lead 12 has a cross-sectional diameter of no more than 1.5 millimeters (1.5 mm) and may be in the range of 0.5 to 1.5 mm. In at least some embodiments, the lead 12 has a length of at least 10 centimeters (10 cm) and the length of the lead 12 may be in the range of 10 to 70 cm.

The electrodes 26 can be made using a metal, alloy, conductive oxide, or any other suitable conductive biocompatible material. Examples of suitable materials include, but are not limited to, platinum, platinum iridium alloy, iridium, titanium, tungsten, palladium, palladium rhodium, or the like. Preferably, the electrodes are made of a material that is biocompatible and does not substantially corrode under expected operating conditions in the operating environment for the expected duration of use. Each of the electrodes can either be used or unused (OFF). When the electrode is used, the electrode can be used as an anode or cathode and carry anodic or cathodic current. In some instances, an electrode might be an anode for a period of time and a cathode for a period of time.

Deep brain stimulation leads and other leads may include one or more sets of segmented electrodes. Segmented electrodes may provide for superior current steering than ring electrodes because target structures in deep brain stimulation or other stimulation are not typically symmetric about the axis of the distal electrode array. Instead, a target may be located on one side of a plane running through the axis of the lead. Through the use of a radially segmented electrode array (“RSEA”), current steering can be performed not only along a length of the lead but also around a circumference of the lead. This provides precise three-dimensional targeting and delivery of the current stimulus to neural target tissue, while potentially avoiding stimulation of other tissue.

Any number of segmented electrodes 530 may be disposed on the lead body 510 including, for example, anywhere from one to sixteen or more segmented electrodes 530. It will be understood that any number of segmented electrodes 530 may be disposed along the length of the lead body 510. A segmented electrode 530 typically extends only 75%, 67%, 60%, 50%, 40%, 33%, 25%, 20%, 17%, 15%, or less around the circumference of the lead.

The segmented electrodes 530 may be grouped into sets of segmented electrodes, where each set is disposed around a circumference of the lead 12 at a particular longitudinal portion of the lead 12. The lead 12 may have any number segmented electrodes 530 in a given set of segmented electrodes. The lead 12 may have one, two, three, four, five, six, seven, eight, or more segmented electrodes 530 in a given set. In at least some embodiments, each set of segmented electrodes 530 of the lead 12 contains the same number of segmented electrodes 530. The segmented electrodes 530 disposed on the lead 12 may include a different number of electrodes than at least one other set of segmented electrodes 530 disposed on the lead 12. The segmented electrodes 530 may vary in size and shape. In some embodiments, the segmented electrodes 530 are all of the same size, shape, diameter, width or area or any combination thereof. In some embodiments, the segmented electrodes 530 of each circumferential set (or even all segmented electrodes disposed on the lead 12) may be identical in size and shape.

Each set of segmented electrodes 530 may be disposed around the circumference of the lead body 510 to form a substantially cylindrical shape around the lead body 510. The spacing between individual electrodes of a given set of the segmented electrodes may be the same, or different from, the spacing between individual electrodes of another set of segmented electrodes on the lead 12. In at least some embodiments, equal spaces, gaps or cutouts are disposed between each segmented electrode 530 around the circumference of the lead body 510. In other embodiments, the spaces, gaps or cutouts between the segmented electrodes 530 may differ in size, or cutouts between segmented electrodes 530 may be uniform for a particular set of the segmented electrodes 530 or for all sets of the segmented electrodes 530. The sets of segmented electrodes 530 may be positioned in irregular or regular intervals along a length the lead body 510.

Conductor wires (not shown) that attach to the ring electrodes 520 or segmented electrodes 530 extend along the lead body 510. These conductor wires may extend through the material of the lead 12 or along one or more lumens defined by the lead 12, or both. The conductor wires couple the electrodes 520, 530 to the terminals 27.

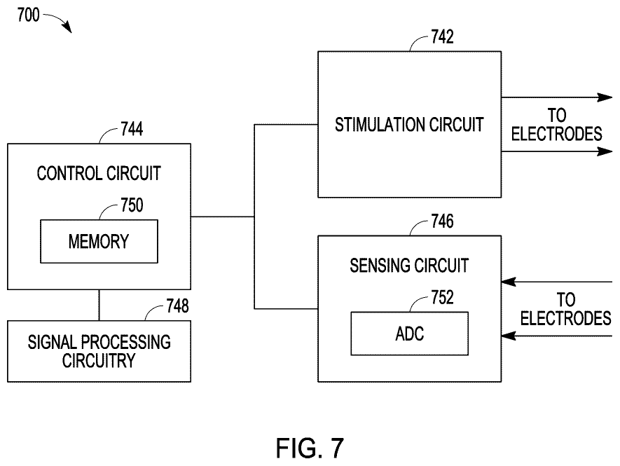

FIGS. 6A-6H are illustrations of different embodiments of leads 12 with segmented electrodes 330, optional ring electrodes 320 or tip electrodes 320a, and a lead body 310. The sets of segmented electrodes 330 each include either two (FIG. 6B), three (FIGS. 6E-6H), or four (FIGS. 6A, 6C, and 6D) or any other number of segmented electrodes including, for example, three, five, six, or more. The sets of segmented electrodes 330 can be aligned with each other (FIGS. 6A-6G) or staggered (FIG. 6H).

When the lead 12 includes both ring electrodes 320 and segmented electrodes 330, the ring electrodes 320 and the segmented electrodes 330 may be arranged in any suitable configuration. For example, when the lead 12 includes two ring electrodes 320 and two sets of segmented electrodes 330, the ring electrodes 120 can flank the two sets of segmented electrodes 330 (see e.g., FIGS. 5, 6A, and 6E-6H, ring electrodes 320 and segmented electrode 330). Alternately, the two sets of ring electrodes 320 can be disposed proximal to the two sets of segmented electrodes 330 (see e.g., FIG. 6C, ring electrodes 320 and segmented electrode 330), or the two sets of ring electrodes 320 can be disposed distal to the two sets of segmented electrodes 330 (see e.g., FIG. 6D, ring electrodes 320 and segmented electrode 330). One of the ring electrodes can be a tip electrode (see e.g., tip electrode 320a of FIGS. 36E and 6G). It will be understood that other configurations are possible as well (e.g., alternating ring and segmented electrodes, or the like).

By varying the location of the segmented electrodes 330, different coverage of the target neurons may be selected. For example, the electrode arrangement of FIG. 6C may be useful if the physician anticipates that the neural target will be closer to a distal tip of the lead body 310, while the electrode arrangement of FIG. 6D may be useful if the physician anticipates that the neural target will be closer to a proximal end of the lead body 310.

Any combination of ring electrodes 320 and segmented electrodes 330 may be disposed on the lead 12. For example, the lead 12 may include a first ring electrode 320, two sets of segmented electrodes; each set formed of four segmented electrodes 330, and a final ring electrode 320 at the end of the lead. This configuration may simply be referred to as a 1-4-4-1 configuration (FIGS. 6A and 6E, ring electrodes 320 and segmented electrode 330). It may be useful to refer to the electrodes with this shorthand notation. Thus, the embodiment of FIG. 6C may be referred to as a 1-1-4-4 configuration, while the embodiment of FIG. 6D may be referred to as a 4-4-1-1 configuration. The embodiments of FIGS. 6F, 6G, and 6H can be referred to as a 1-3-3-1 configuration. Other electrode configurations include, for example, a 2-2-2-2 configuration, where four sets of segmented electrodes are disposed on the lead, and a 4-4 configuration, where two sets of segmented electrodes, each having four segmented electrodes 330 are disposed on the lead. The 1-3-3-1 electrode configuration of FIGS. 6F, 6G, and 6H has two sets of segmented electrodes, each set containing three electrodes disposed around the circumference of the lead, flanked by two ring electrodes (FIGS. 6F and 6H) or a ring electrode and a tip electrode (FIG. 6G). In some embodiments, the lead includes 16 electrodes. Possible configurations for a 16-electrode lead include but are not limited to 4-4-4-4; 8-8; 3-3-3-3-3-1 (and all rearrangements of this configuration); and 2-2-2-2-2-2-2-2.

Any other suitable arrangements of segmented and/or ring electrodes can be used. As an example, arrangements in which segmented electrodes are arranged helically with respect to each other. One embodiment includes a double helix. One or more electrical stimulation leads can be implanted in the body of a patient (for example, in the brain or spinal cord of the patient) and used to stimulate surrounding tissue. The lead(s) are coupled to the implantable pulse generator (such as IPG 14 in FIG. 2).

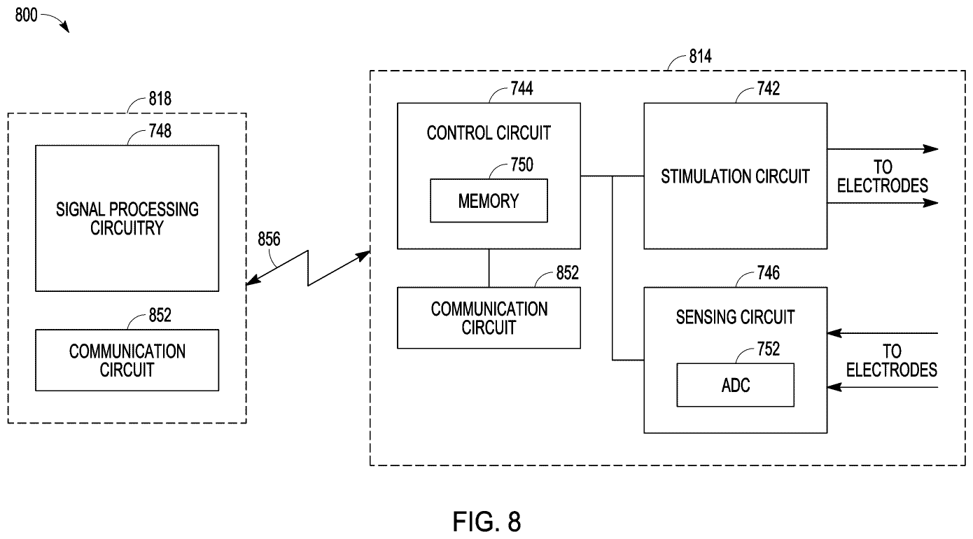

FIG. 7 is a block diagram of portions of an embodiment of a neurostimulation system 700 for providing neurostimulation to a patient. The neurostimulation system 700 includes a stimulation circuit 742, a control circuit 744, and a sensing circuit 746. The stimulation circuit 742 can be operatively coupled to stimulation electrodes such as any of the electrodes described herein and the stimulation circuit 742 provides or delivers electrical neurostimulation energy to the electrodes. The sensing circuit 746 can include one or more sense amplifiers switchable among recording electrodes to sense internal neural signals of the patient. The control circuit 744 can include a processor such as a microprocessor, a digital signal processor, application specific integrated circuit (ASIC), or other type of processor, interpreting or executing instructions in software modules or firmware modules. The instructions can be stored in memory 750 that can be integral to the control circuit 744 or separate from the control circuit 744. The control circuit 744 can include other circuits or sub-circuits to perform the functions described. These circuits may include software, hardware, firmware, or any combination thereof. Multiple functions can be performed in one or more of the circuits or sub-circuits as desired.

The neurostimulation system 700 includes signal processing circuitry 748 that can be separate from the control circuit 744 or integral to the control circuit 744. The signal processing circuitry 748 can include one or more processes running on one or processors (e.g., microprocessors) to perform signal analysis or other signal processing on the neural signals sensed using the sensing circuit 746. The signal processing circuitry can include an analog-to-digital converter (ADC) circuit 752 to digitize the sensed neural signals for signal processing.

The neurostimulation system 700 is connectable to at least one stimulation lead (e.g., stimulation lead 12 in FIG. 5) that can be implanted in the body of a patient (for example, in the brain of the patient) and the electrodes of the stimulation lead is used to stimulate surrounding tissue. The control circuit 744 can initiate delivery of electrical neurostimulation using the stimulation circuit 742. The electrical neurostimulation may be delivered according to neurostimulation therapy parameters configured in the control circuit 744 (e.g., by programming). The neurostimulation system 700 can sense electrical neural signals when coupled to the electrodes. The neurostimulation system 700 can be used at the time of implanting or sometime after implanting to sense the response of the patient to neurostimulation. The sensed response can be used to adjust the neurostimulation by either an automatic adjustment of the parameters of the neurostimulation or by recommending parameter settings to a user.

The neurostimulation system 700 can be included in one device or in multiple devices. For a one device example, the stimulation circuit 742, sensing circuit 746, control circuit 744, and signal processing circuitry 748 can be included in an external neurostimulator (e.g., ETS 20 in FIG. 2) or in an internal neurostimulator (e.g., IPG 14 in FIG. 2).

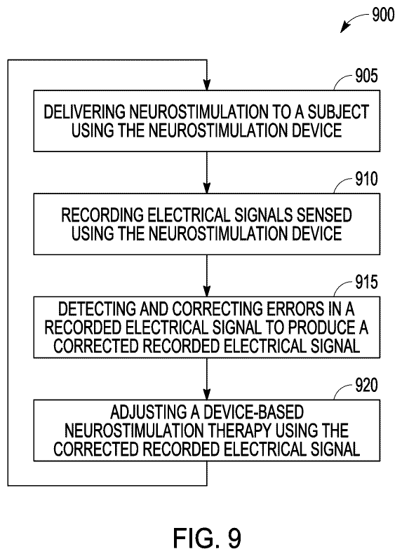

FIG. 8 is a block diagram of another example of a multi-device neurostimulation system 800. The neurostimulation system 800 can include an IPG 814 (e.g., IPG 14 in FIG. 2) and an IPG programming device 818 (e.g., CP 18 in FIG. 2). The stimulation circuit 742, sensing circuit 746, and control circuit 744 are included in the IPG 814, and the signal processing circuitry 748 is included in the programming device 818 (e.g., CP 18 in FIG. 2). The IPG 814 and programming device 818 both include communication circuits 852 that transfer information using wireless signals (856). The programming device 818 programs neurostimulation therapy parameters into the IPG 814 using the communication circuits.

The human nervous system produces a neural response to neurostimulation received via sensory receptors or received directly into any part of the network of neural elements that forms the nervous system. Additionally, neurostimulation can excite elements of the human nervous system in a manner that produces neural responses. These neural responses are known as evoked potentials and the stimulation that evokes these responses can be called evoking neurostimulation.

Evoked potential (EP) signals can be sensed electrically by the neurostimulation system, such as by using sense amplifiers of the sensing circuit 746 coupled to recording electrodes for example. A single or repetitive stimulus can be applied or presented to the nervous system and the evoked potential signals (e.g., evoked resonant neural activity (ERNA) signals) can be sensed using sensing amplifiers of the sensing circuit 746. The electrical signals output from the sensing amplifiers can be digitized using an ADC 752. The digitized electrical signals can be recorded (e.g., stored in memory) and processed using the signal processing circuitry 748 (e.g., filtered by averaging) to detect presence of evoked potentials. The processed recorded electrical signals can be input to therapy decision-making algorithms performed by the neurostimulation system.

The decision-making algorithms and the sub-components of the algorithm (e.g., signal processing steps) rely on accurate data to output an appropriate decision (e.g., a device-based decision to adjust one or more parameters of the neurostimulation therapy). However, system-level problems can affect the accuracy of the recorded signal data. For example, dropped packets communicated between devices or missing ADC codes can result in missing evoked potential signal data. In another example, one or more of system noise, physiological noise, and environmental noise can result in incorrect evoked potential signal data. The incorrect data or missing data may cause a decision-making algorithm to output a non-optimal decision. To improve the decision-making by the neurostimulation systems, the signal processing circuitry 748 can detect and correct errors in the recorded electrical signals.

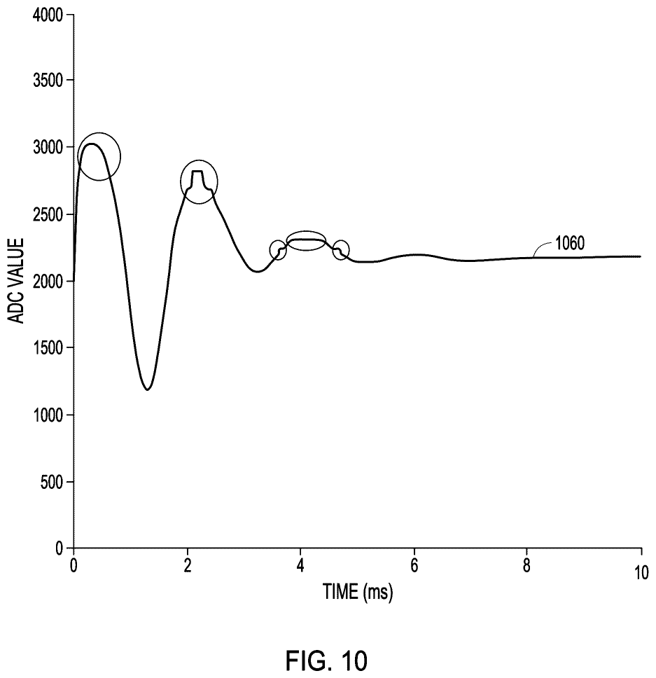

FIG. 9 is a flow diagram of an example of a method 900 of operating a neurostimulation system to deliver electrical neurostimulation to a patient. The system includes a stimulation circuit (e.g., stimulation circuit 742 in FIG. 7 or FIG. 8) operatively coupled to a stimulation lead (e.g., stimulation lead 12 in FIG. 2) that is implantable in a patient or subject. The stimulation lead can include multiple electrodes, including one or both of ring electrodes and segmented electrodes.

At block 905, neurostimulation energy is delivered to the patient or subject using the neurostimulation system (e.g., using the stimulation circuit 742 in FIG. 7 or FIG. 8). The timing of the neurostimulation delivery can be controlled using a control circuit of the neurostimulation device (e.g., control circuit 744 of FIG. 7 or FIG. 8). At block 910, electrical signals are sensed and recorded using the neurostimulation device. The electrical signals are sensed using a signal sensing circuit (e.g., sensing circuit 706 in FIG. 7 or FIG. 8). The sensed electrical signals are recorded such as by digitizing the sensed electrical signals using an ADC (e.g., ADC 752 in FIG. 7 or FIG. 8) and storing the sensed electrical signals in the memory of the neurostimulation device (e.g., memory 750 in FIG. 7 or FIG. 8). The recorded electrical signals may be the signals resulting from evoking neurostimulation delivered by the neurostimulation device.

The recorded electrical signals may include evoked response signals (e.g., ERNA signals and DLEP signals) or local field potentials (LFPs). These signals may input to decision-making algorithms performed by a neurostimulation device or a programmer for the neurostimulation device. The signals can be used by the neurostimulation device to score, recommend, or determine appropriate stimulation parameters (e.g., the set of electrodes used to deliver current, the polarity and relative strength of same, the magnitude, the amplitude, pulse-width, rate, or pattern of stimulation, etc.) to customize neurostimulation for the patient (including, updating or adjusting stimulation parameters, including intermittently or continuously, including when the initialization or update is done with a programming person or in an semi or fully automated or autonomous fashion), or to provide recommendations regarding lead positioning for the neurostimulation. If the recorded electrical signals have lower accuracy, the neurostimulation or lead positioning derived by the neurostimulation device may be less effective.

To improve accuracy of electrical signal data input to the device-based decision-making algorithms, at block 915, errors in the recorded electrical signals to produce corrected recorded electrical signals are detected and corrected using the signal processing circuitry of the neurostimulation system (e.g., the signal processing circuitry 748 in FIG. 7 or FIG. 8). At block 920, the neurostimulation system adjusts the device-based neurostimulation therapy provided to the subject using the corrected recorded electrical signal (e.g., setting appropriate values of stimulation parameters). The method 900 can be a closed loop process with the method 900 returning to recording additional electrical signals and making adjustments to the neurostimulation based on findings in the recorded electrical signals.

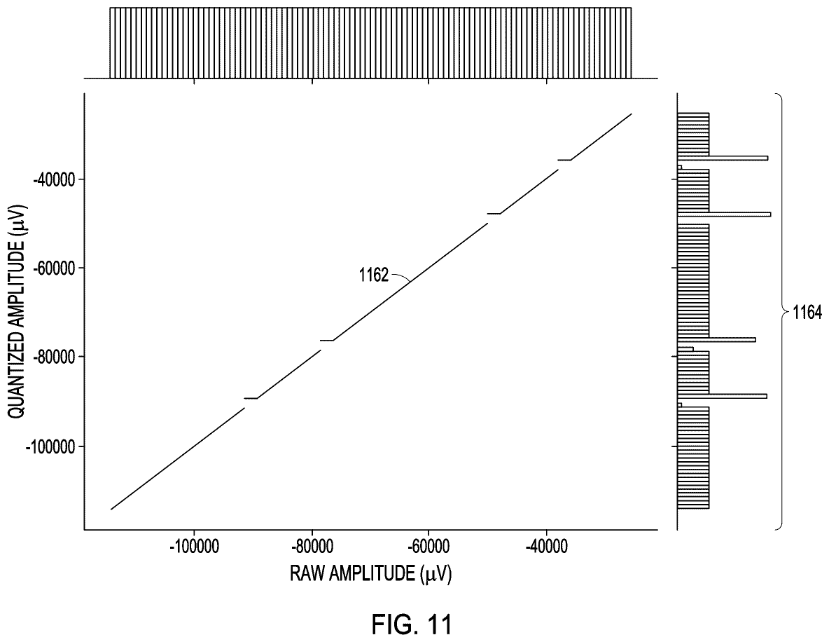

FIG. 10 is an illustration of an example of an electrical signal 1060 recorded by the neurostimulation system. The electrical signal 1060 may be a raw recorded electrical signal that was sensed and digitized but hasn't been processed by the signal processing circuitry. The electrical signal 1060 includes incorrect data and the circles in the Figure indicate where the signal data is incorrect. As shown in FIG. 10, the signal portions within the circles have a slope that changes abruptly. The abrupt slope changes in a recorded electrical signal can be caused by discontinuities in quantizing or digitizing the signal output from the sense amplifiers. This may be particularly true for LFP signals, and the quickly changing slope give the signals a noise-like appearance.

The raw recorded electrical signal is input into the signal processing circuitry, and the signal processing circuitry detects the incorrect data in the raw recorded electrical signal and removes the incorrect data from the raw recorded electrical signal to produce a corrected recorded electrical signal. In some examples, the signal processing circuitry performs a signal smoothing algorithm on the recorded electrical signal to produce the corrected recorded electrical signal. The signal smoothing algorithm replaces the incorrect signal data with signal data to smooth the abrupt slope changes in the recorded electrical signal.

In the case where the signal of interest is an LFP, the character of the signal is more random. Repeated data points in the signal may be indicative of recording error by the neurostimulation system. To correct data in LFP signals, the data correction algorithm may rely on interpolation and overlay of gross estimated signal characteristics, such as could be derived from the power spectral density or fast Fourier transform of an LFP signal.

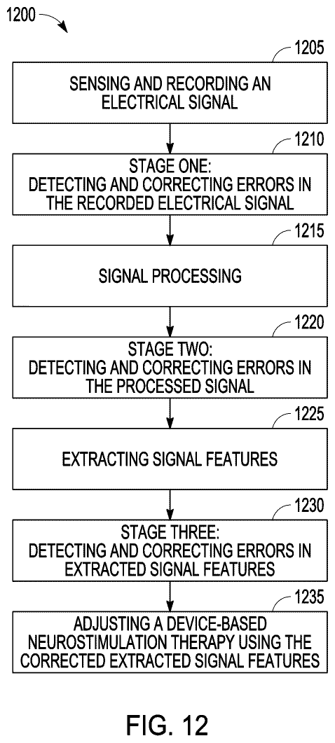

FIG. 11 shows an example of a transfer function 1162 of an ADC. Transfer function quantizes the input signal amplitude of the horizontal axis at the bottom of FIG. 11 to the quantized signal amplitude of the vertical axis at the left of FIG. 11. The transfer function 1162 shows four discontinuities in the transfer function. The vertical axis on the right in FIG. 11 shows the abrupt changes in quantization 1164 that can be caused by the discontinuities in the transfer function 1162. The signal processing circuitry may apply a smoothing algorithm to smooth the discontinuities in the recorded electrical signal. In another approach, values of the discontinuities in the transfer function of the ADC are known and errors in the ADC are pre-characterized. The signal processing circuitry replaces the digital output value of the ADC with a different digitized value to smooth the raw recorded digitized signal to produce a corrected recorded electrical signal.

In some examples, the signal processing circuitry detects missing data in the recorded electrical signal. Data in the recorded signal may be missing because of having been dropped for example if the raw recorded data is transferred to a separate device (e.g., a neurostimulation device programmer) for signal processing. The signal processing circuitry performs a data interpolation algorithm to include the missing data or estimate the missing data to produce corrected recorded electrical signals. Some examples of the data interpolation algorithm include an expectation-maximization (EM) algorithm, a maximum-likelihood estimation (MLE) algorithm, and a Kalman smoothing algorithm. In some examples, the signal processing circuitry low pass filters the recorded electrical signal to estimate the missing data and produce the corrected recorded electrical signal.

The corrected recorded electrical signal can be used for signal feature extraction (e.g., by the signal processing circuitry). Examples of the signal features include the greatest value of the magnitude of the signals, the amplitude of a detected peak in the signals (e.g., first negative peak, first positive peak, or other peak in the signals), the time of the detected peak, phase of the signals, frequency of the signals, etc. Other features, combinations of features, and sets of features can be used. The extracted features can be used for decision making by the neurostimulation system.

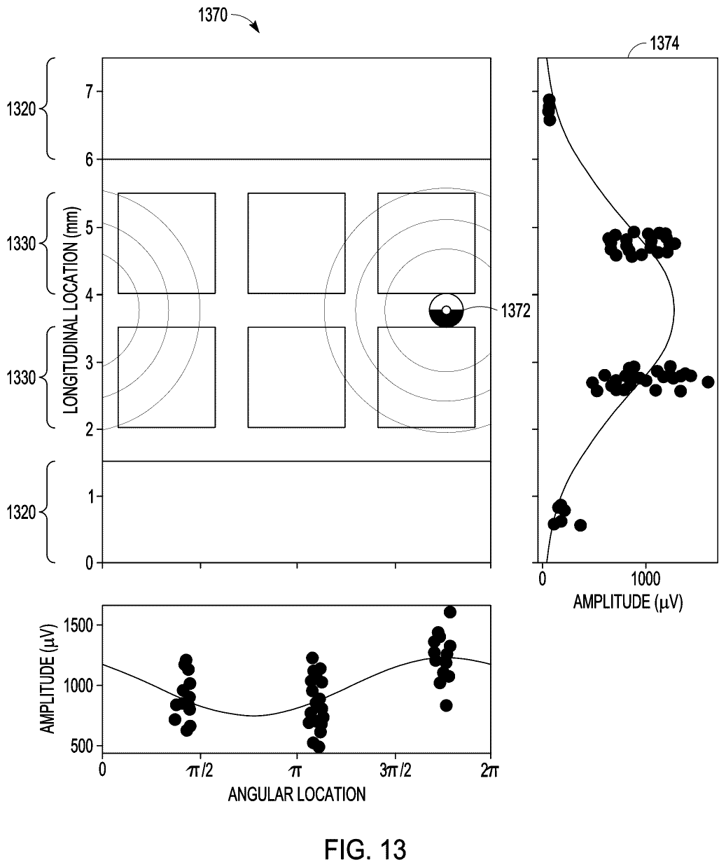

FIG. 12 is a flow diagram of another example of a method 1200 of operating a neurostimulation system to deliver electrical neurostimulation to a patient. The method 1200 may be performed by a neurostimulation device of the system (e.g., a an external neurostimulator (e.g., ETS 20 in FIG. 2) or an internal neurostimulator (e.g., IPG 14 in FIG. 2), or a programming device of the system (e.g., CP 18 in FIG. 2). The method 1200 includes multiple stages of error detection and correction to address signal artifacts that may inadvertently be added at different stages of processing by the system. The stages of error detection and correction may be optional, and implementations may include any one or more of the error detection and correction stages.

At block 1205, electrical signals are sensed and recorded using the neurostimulation system. Neurostimulation energy (e.g., evoking neurostimulation energy) may be provided using the neurostimulation system to produce evoked response signals as the sensed electrical signals. At block 1210, a first stage of error detection and correction is applied to the recorded electrical signals using signal processing circuitry to detect and correct errors in the recorded electrical signals. The error detection may include signal slope or smoothness analysis by the signal processing circuitry to detect abrupt changes in the recorded electrical signal. In some examples, the signal processing circuitry may perform an analysis in the frequency domain to detect signal components having a signal frequency associated with a glitch signal frequency.

Error correction may include the signal processing circuitry performing an interpolation algorithm to interpolate missing or incorrect data in the recorded electrical signals. The interpolation algorithm may enforce first order (e.g., linear) smoothness, or a higher order (e.g., nth order) smoothness. In some examples, the signal processing circuitry uses characterization of the ADC to correct abrupt changes in the ADC output. The characterization may include correction data. The signal processing circuitry may replace recorded signal data with the correction data for the ADC outputs characterized as not desired. In certain examples, the signal processing circuitry determines the correction data using a lookup table indexed by the ADC data to be replaced. The result of the Stage One error detection and correction is a corrected recorded electrical signal.

At block 1215, the neurostimulation system may perform other signal processing on the corrected recorded electrical signal to produce a processed corrected recorded electrical signal. For example, the signal processing circuitry may filter the corrected recorded electrical signal to produce a filtered corrected recorded electrical signal, or the signal processing circuitry may amplify the corrected recorded electrical signal. This may be useful to help identify a particular signal feature of interest. At block 1220, a second stage of error detection and correction is applied to the processed corrected recorded electrical signals. For example, the filtering may add a filter-related artifact (e.g., a phase shift) that can be corrected by the Stage Two error detection and correction.

At block 1225, the signal processing circuitry extracts one or more signal features from the corrected recorded electrical signals. Some examples of the signal features that can be extracted by the signa processing circuitry include the greatest value of the magnitude of the signals, the amplitude of a detected peak in the signals (e.g., first negative peak, first positive peak, or other peak in the signals), the time of the detected peak, phase of the signals, frequency of the signals, etc. Other features, combinations of features, and sets of features could be extracted by the signal processing circuitry.

At block 1230, a third stage of error detection and correction is applied to the extracted signal features. The signal processing circuitry may detect errors in the extracted signal features. The errors may be detected using a statistical analysis of what features are expected in the recorded electrical signals. For example, an expected statistical standard deviation for a signal feature may be defined a priori. The signal processing circuitry determines if the signal feature is within the standard deviation defined for the feature. If the extracted signal feature is within the standard deviation, the extracted signal feature is input into the decision-making algorithm performed by the neurostimulation system. For example, the signal processing circuitry may use the extracted signal feature to identify an evoked response signal of interest from among the recorded electrical signals.

If the extracted signal feature is not within the standard deviation, the recorded electrical signal may be discarded and not used by the decision-making algorithm. In some examples, the signal feature is removed from the recorded electrical signal and the corrected recorded electrical signal with the signal feature removed is used by the decision-making algorithm. In some examples, the signal processing circuitry corrects the error in the extracted signal feature, and the corrected recorded electrical signal with the signal feature corrected is used by the decision-making algorithm.

At block 1235, the control circuit may use the corrected recorded electrical signal with the signal feature with the extracted signal feature removed or corrected to determine or adjust a value of a neurostimulation therapy parameter. For example, the neurostimulation system may set a stimulation configuration of the neurostimulation to the stimulation configuration that produced the highest amplitude evoked response signal. The optimized stimulation configuration may include a stimulation energy and an electrode combination that produced the highest amplitude evoked response signal. In some examples, the extracted signal features can be used to produce a recommendation regarding placement of the neurostimulation lead or lead electrodes to use for the stimulation.

The signal processing circuitry of the system may use the extracted features to create a hotspot view or hotspot fit of the sensed recorded signals. The sensed recorded signal may be evoked response signals and the hotspot view may be used to show the results of stimulation steering, with the “hotspot” showing the best results defined for the study.

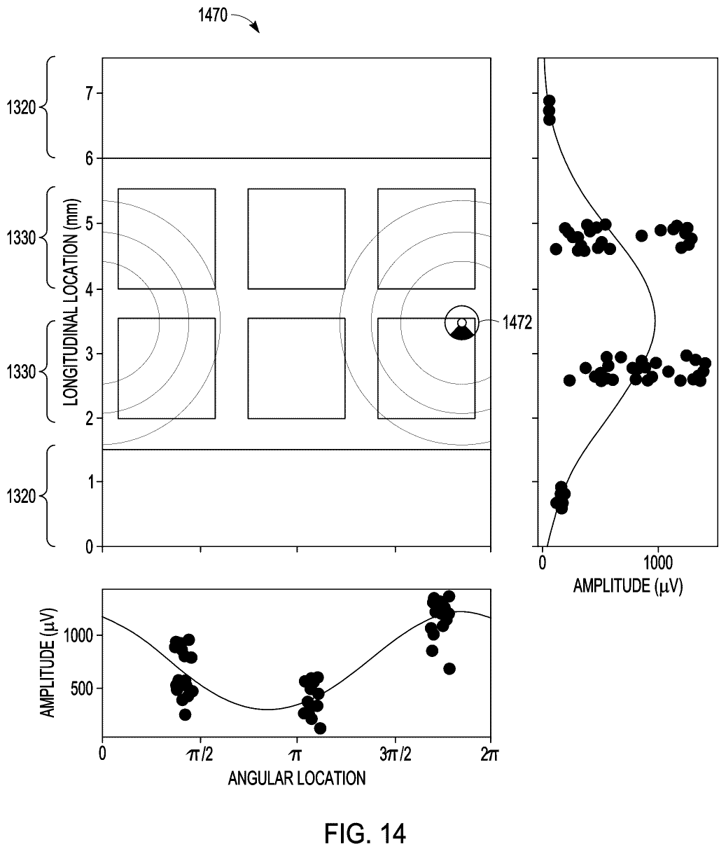

FIGS. 13 and 14 illustrate examples of a hotspot view that can be produced by a neurostimulation system. In FIG. 13, the hotspot view 1370 is shown superimposed on an electrode template representing electrodes of the lead, including ring electrodes 1320, and segmented electrodes 1330. The hotpot view 1370 created by the signal processing circuitry can be displayed on a user interface (e.g., a user interface of a CP 18 in FIG. 2). The hotpot view 1370 may be color coded similar to thermal mapping, where more signal features in a particular area result in a “hotter” color for the area. The area with the most features may be displayed as a hotspot indicator 1372. (e.g., a highlight or icon).

FIG. 13 also shows distributions for the extracted signal features computed by the signal processing circuitry. In the Example of FIG. 13, the extracted feature is amplitude, and the measured amplitudes are shown as dots in the distributions. In the distribution on the right 1374, the measured amplitudes are shown with the vertical or length location on the lead used for the stimulation steering that resulted in the measurement. In the distribution on the bottom 1376, the measured amplitudes are shown with the angular location on the lead used for the stimulation steering. The hotspot indicator 1372 may correspond to peak regions of the computed distributions. The hotspot indicator 1372 may be positioned to represent the length location and the angular location of the peak regions on the length of the lead. Thus, the hotspot indicator 1372 can correspond to the best stimulation steering setting for the patient as determined by the neurostimulation system.

Errors in the recorded electrical signals used to create the hotspot view may result in incorrect results for the hotspot view. FIG. 14 represents a hotspot view 1470 determined with the error detection and correction stages in FIG. 12. The error correction of the recorded electrical signals may result in the hotspot indicator 1472 corresponding to more accurate length and angular locations of the peak region of the signal feature.

The systems and methods described herein include techniques for pre-processing of neural signal data that can improve the results of device-based adaptive or decision-making algorithms for optimizing neurostimulation therapy. The embodiments described herein can be methods that are machine or computer-implemented at least in part. Some embodiments may include a computer-readable medium or machine-readable medium encoded with instructions operable to configure an electronic device or system to perform methods as described in the above examples. An implementation of such methods can include code, such as microcode, assembly language code, a higher-level language code, or the like. Such code can include computer readable instructions for performing various methods.

The code can form portions of computer program products. Further, the code can be tangibly stored on one or more volatile or non-volatile computer-readable media during execution or at other times. Various embodiments are illustrated in the figures above. One or more features from one or more of these embodiments may be combined to form other embodiments.

The above detailed description is intended to be illustrative, and not restrictive. The scope of the disclosure should, therefore, be determined with references to the appended claims, along with the full scope of equivalents to which such claims are entitled.

What is claimed is:

1. A computer-implemented method of operating a neurostimulation system, the method comprising:
delivering neurostimulation to a subject using a neurostimulation device of the neurostimulation system when the neurostimulation device is connected to an implantable stimulation lead;
recording electrical signals sensed using the implantable stimulation lead;
detecting and correcting errors in a recorded electrical signal to produce a corrected recorded electrical signal; and
adjusting a device-based neurostimulation therapy provided to the subject using the corrected recorded electrical signal.

2. The method of claim 1, wherein the detecting and correcting errors in the recorded electrical signal includes at least one of the neurostimulation device and a programmer of the neurostimulation device detecting incorrect data and removing the incorrect data from the recorded electrical signal.

3. The method of claim 2, wherein the removing the incorrect data includes the at least one of the neurostimulation device and the programmer applying a signal smoothing algorithm to the recorded electrical signal.

4. The method of claim 1, wherein the detecting and correcting errors in the recorded electrical signal includes at least one of the neurostimulation device and a programmer of the neurostimulation device detecting missing data in the recorded electrical signal and producing the missing data using a data interpolation algorithm.

5. The method of claim 4, wherein the producing the missing data using includes the at least one of the neurostimulation device and the programmer producing the missing data using one or more of an expectation-maximization (EM) algorithm, a maximum-likelihood estimation (MLE) algorithm, a Kalman smoothing algorithm, or low pass filtering of the recorded electrical signal.

6. The method of claim 1, including:
filtering the corrected recorded electrical signal, using at least one of the neurostimulation device and a programmer for the neurostimulation device, to produce a filtered corrected recorded electrical signal; and
detecting and correcting a signal artifact in the filtered corrected recorded electrical signal introduced by the filtering.

7. The method of claim 1, including:
extracting one or more features from the corrected recorded electrical signal;
detecting an error in the one or more extracted features of the corrected recorded electrical signal; and
removing the one or more extracted features having the detected error or correcting the error in the one or more extracted features.

8. The method of claim 1, including:
extracting one or more features from the corrected recorded electrical signal; and
identifying, by at least one of the neurostimulation device and a programmer for the neurostimulation device, an evoked response signal of interest from among the recorded electrical signals using the one or more extracted features of the corrected recorded electrical signal.

9. A programming device for a neurostimulation device that provides electrical neurostimulation to a subject according to at least one neurostimulation therapy parameter when coupled to an implantable stimulation lead, the programming device comprising:
a communication circuit configured to receive a recorded electrical signal from the neurostimulation device and send the at least one therapy parameter to the neurostimulation device; and
signal processing circuitry configured to:
detect and correct errors in the recorded electrical signal to produce a corrected recorded electrical signal; and
determine a value of the at least one therapy parameter using the corrected recorded electrical signal.

10. The programming device of claim 9, wherein the signal processing circuitry is configured to detect incorrect data in the recorded electrical signal and remove the incorrect data from the recorded electrical signal to produce the corrected recorded electrical signal.

11. The programming device of claim 10, wherein the signal processing circuitry is configured to perform a signal smoothing algorithm on the recorded electrical signal to produce the corrected recorded electrical signal.

12. The programming device of claim 9, wherein the signal processing circuitry is configured to detect missing data in the recorded electrical signal and perform a data interpolation algorithm to include the missing data in the corrected recorded electrical signal.

13. The programming device of claim 12, wherein the data interpolation algorithm includes at least one of an expectation-maximization (EM) algorithm, a maximum-likelihood estimation (MLE) algorithm, a Kalman smoothing algorithm, or low pass filtering of the recorded electrical signal.

14. The programming device of claim 9, wherein the signal processing circuitry is configured to:
filter the corrected recorded electrical signal to produce a filtered corrected recorded electrical signal; and
detect and correct a signal artifact in the filtered corrected recorded electrical signal introduced by the filtering.

15. The programming device of claim 9, wherein the signal processing circuitry is configured to:
extract one or more features from the corrected recorded electrical signal;
detect an error in the one or more extracted features of the corrected recorded electrical signal; and
remove the one or more extracted features having the detected error or correct the error in the one or more extracted features.

16. The programming device of claim 9, wherein the signal processing circuitry is configured to:
extract one or more features from the corrected recorded electrical signal; and
identify an evoked response signal of interest from among the recorded electrical signals using the one or more extracted features of the corrected recorded electrical signal.

17. A neurostimulation device comprising:
a stimulation circuit configured to deliver electrical neurostimulation to a subject when coupled to an implantable stimulation lead;
a sensing circuit configured to sense electrical signals when coupled to the stimulation lead;
a control circuit operatively coupled to the stimulation circuit and the sensing circuit, and configured to initiate delivery of neurostimulation to the subject and record sensed electrical signals resulting from the neurostimulation; and
signal processing circuitry configured to detect and correct errors in a recorded electrical signal to produce a corrected recorded electrical signal;
wherein the control circuit is configured to adjust a device-based neurostimulation therapy provided to the subject using the corrected recorded electrical signal.

18. The neurostimulation device of claim 17, wherein the signal processing circuitry is configured to detect incorrect data in the recorded electrical signal and remove the incorrect data from the recorded electrical signal to produce the corrected recorded electrical signal.

19. The neurostimulation device of claim 17, wherein the signal processing circuitry is configured to detect missing data in the recorded electrical signal and perform a data interpolation algorithm to include the missing data in the corrected recorded electrical signal.

20. The neurostimulation device of claim 17, wherein the signal processing circuitry is configured to:
extract one or more features from the corrected recorded electrical signal; and
identify an evoked response signal of interest from among the recorded electrical signals using the one or more extracted features of the corrected recorded electrical signal.
